(19) United States
(12) Patent Application Publication
(10) Pub. No.: US 2005/0090548 A1
(43) Pub. Date: Apr. 28, 2005

(54) Intrathecal gabapentin for treatment of epilepsy

(75) Inventors: Keith R. Hildebrand, Houlton, WI (US); Linda M. Page, Woodbury, MN (US); Deanna S. Lane, Columbia Heights, MN (US); Dennis D. Elsberry, Plymouth, MN (US); David A. Clarahan, Blaine, MN (US); Jayantha H. Ratnayake, Blaine, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc., Minneapolis, MN (US)

(21) Appl. No.: 10/807,827

(22) Filed: Mar. 24, 2004

Related U.S. Application Data
(60) Provisional application No. 60/513,681, filed on Oct. 23, 2003.
(60) Provisional application No. 60/513,682, filed on Oct. 23, 2003.

(51) Int. Cl. A61K 31/195; A61M 31/00

(52) U.S. Cl. 514/561; 604/500

(57) ABSTRACT

Methods for treating epilepsy by administering gabapentin to cerebrospinal fluid and brain tissue of a patient are discussed. Compositions, particularly injectable compositions, containing gabapentin are also discussed. In addition, systems including an implantable device having a pump coupled to a reservoir for housing a composition, a catheter having a proximal end coupled to the pump and having a distal end adapted for administrating a composition to a cerebrospinal fluid of a patient, and a composition containing gabapentin, which composition is housed in the reservoir of the pump, are also discussed.

### RELATED APPLICATIONS

This application claims priority to Provisional Application Ser. No. 60/513,682, entitled “INTRATHECAL GABAPENTIN FOR TREATMENT OF PAIN AND EPILEPSY”, filed on Oct. 23, 2003, and Provisional Application Ser. No. 60/513,681, entitled “INTRATHECAL GABAPENTIN FOR TREATMENT OF PAIN AND EPILEPSY”, filed on Oct. 23, 2003. Each of the above-referenced applications is herein incorporated by reference in their entirety.

### FIELD OF THE INVENTION

This invention relates to medical devices, therapeutic methods, and compositions for delivering gabapentin to a patient.

### BACKGROUND

Epilepsy is a condition associated with recurrent seizures that affects people throughout the world, including more than 1 million people in the United States alone. In the US, epilepsy occurs with a prevalence of about 5 people per 1,000 with some countries reporting an even higher prevalence. Epileptic seizures are the outward manifestation of excessive and/or hyper-synchronous abnormal activity of neurons in the cerebral cortex. Seizures are usually self-limiting and can be of many types. The behavioral manifestations of a seizure reflect the functions of the cortical region where the hyper activity is occurring. Seizures can be generalized, appearing to involve the entire brain simultaneously. Some types of seizures, partial seizures, begin in one part of the brain and remain local. While rarely fatal, epilepsy has been associated with an increased risk of mortality.

Many people require therapeutic intervention for extended periods of time, or even their entire life, to avoid the disruptive and potentially dangerous consequences of seizures. Various pharmacological agents have been used for treatment of epilepsy, including traditional anticonvulsants such as phenyloin, phenobarbital, primidone, carbamazepine, ethosuximide, clonazepam, valproate, and the like. Newer anticonvulsants approved for treating epilspsy include lamotrigine, feribamate, topiramate and gabapentin. While many of these pharmacological agents can eliminate or attenuate the severity of seizures, they may not always be completely effective, and as many as 30% of people may not respond to drug therapy. In addition, their use may be associated with side effects that may become pronounced with long-term administration. Many of the side effects or much of the incomplete efficacy of currently available oral medications for treatment of epilspsy may be due to systemic effects and limited availability to the central nervous system (CNS).

Gabapentin is one pharmacological agent approved for treating epilepsy and has limited access to the CNS. Gabapentin produces a GABA-like inhibitory effect on selective neuronal populations and thus has been useful in treating epilepsy, a disease characterized by hyperactivity of central neurons. Currently only oral formulations of gabapentin are available. However, because orally ingested gabapentin is transported across the gut and the blood-brain barrier via an active and saturable L-amino acid transporter, the amount of gabapentin reaching the CNS sites of action is limited. Because this transporter is saturable, even if the concentration of gabapentin in the plasma is increased, the amount that crosses the blood-brain barrier will remain constant. However, higher CNS levels of gabapentin may be achievable by infusing gabapentin directly into the intrathecal space which bypasses the blood-brain barrier. Intrathecal delivery of gabapentin may be associated with greater efficacy and potentially less side effects. Yet, the use of intrathecally administered gabapentin has not previously been described.

### SUMMARY OF THE INVENTION

An embodiment of the invention provides a system for delivering gabapentin directly to a patient's central nervous system (CNS). The system comprises an amount of gababemtin effective to treat epilepsy when administered directly to the CNS, an implantable pump housing the gabapentin, and a catheter coupled to the pump and adapted to deliver the gabapentin to the patient's CNS. In an embodiment, the system provides for delivery of gabapentin to cerebrospinal fluid of a patient. In an embodiment, the system provides for delivery of gabapentin to brain tissue of a patient.

An embodiment of the invention provides a method for treating epilepsy in a patient in need thereof. The method comprises administering gabapentin to the patient's CNS. In an embodiment, the gabapentin is administered to the patient's CNS by way of an implantable pump system. In an embodiment, the gabapentin is administered to a patient's cerebrospinal fluid. Gabapentin may be administered to the cerebrospinal fluid by infusing gabapentin into the subarachnoid space around the spinal cord, intracerebroventricularly, or through any other medically acceptable route. In an embodiment the gabapentin is delivered directly to a patient's brain tissue, preferably near an epileptic focus.

Advantages of embodiments of the invention include greater control of CNS concentrations of gabapentin, higher levels of gabapentin achievable in the CNS because the blood-brain barrier is bypassed, improved efficacy of gabapentin for treatment of epilepsy, and potential for reduced side effects relative to oral gabapentin. These and other advantages of the invention will become evident upon reading the description herein.

### BRIEF SUMMARY OF THE DRAWINGS

FIG. 1 is a diagrammatic illustration of a patient's brain, the associated spaces containing cerebrospinal fluid, and the flow of cerebrospinal fluid in the subarachnoid space.

FIG. 2 is a diagrammatic illustration of a system for delivering a composition comprising a therapeutic agent according to an embodiment of the present invention.

FIG. 3 is a diagrammatic illustration of a catheter implanted in a patient according to an embodiment of the present invention.

FIG. 4 is a diagrammatic illustration of a catheter implanted in a patient according to an embodiment of the present invention.

FIG. 5 is a diagrammatic illustration of a system including a patient-controlled activator in accordance with an embodiment of the present invention.

The drawings are not necessarily to scale. Like numbers refer to like parts or steps throughout the drawings.

### DETAILED DESCRIPTION

In the following descriptions, reference is made to the accompanying drawings that form a part hereof, and in which are shown by way of illustration several specific embodiments of the invention. It is to be understood that other embodiments of the present invention are contemplated and may be made without departing from the scope or spirit of the present invention. The following detailed description, therefore, is not to be taken in a limiting sense.

All scientific and technical terms used in this application have meanings commonly used in the art unless otherwise specified. The definitions provided herein are to facilitate understanding of certain terms used frequently herein and are not meant to limit the scope of the present disclosure.

In the context of the present invention, the terms “treat”, “therapy”, and the like are meant to include methods to alleviate, slow the progression, prevent, attenuate, or cure the treated disease.

Therapeutic agents referred to herein by name include salts, polymorphs, hydrates, solvates, and the like thereof.

Cerebrospinal Fluid

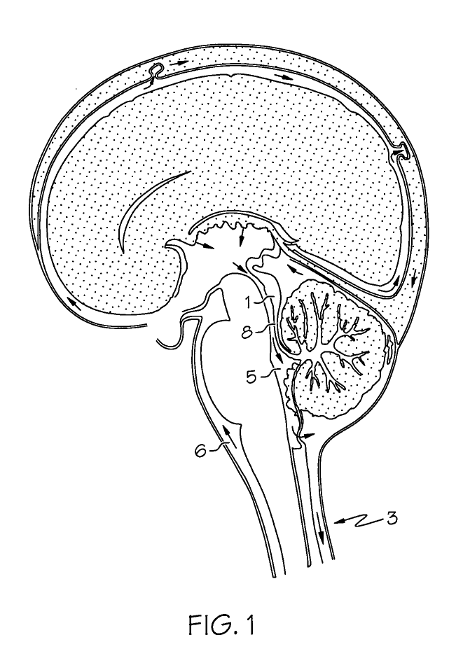

According to an embodiment of the invention, a composition comprising gabapentin may be delivered directly to cerebraspinal fluid 6 of a patient. Referring to FIG. 1, cerebrospinal fluid (CSF) 6 exits the foramen of Magendie and Luschka to flow around the brainstem and cerebellum. The arrows within the subarachnoid space 3 in FIG. 1 indicate cerebrospinal fluid 6 flow. The subarachnoid space 3 is a compartment within the central nervous system that contains cerebrospinal fluid 6. The cerebrospinal fluid 6 is produced in the ventricular system of the brain and communicates freely with the subarachnoid space 3 via the foramen of Magendie and Luschka. A composition comprising gabapentin may be delivered to cerebrospinal fluid 6 of a patient anywhere that the cerebrospinal fluid 6 is accessible.

According to an embodiment of the invention, a composition comprising gabapentin may be administered intrathecally to a patient. Intrathecal delivery of therapeutics into the cerebrospinal fluid 6 can be less invasive than intraparenchymal (direct tissue) delivery of therapeutics. In addition, intrathecal delivery of therapeutics may not require the need for a neurosurgeon as intrathecal delivery of therapeutics does not require delivery to a direct brain target. Other physicians may be qualified to insert a catheter into the subarachnoid space 3 of the spinal column in order to initiate intrathecal therapeutic delivery.

According to an embodiment of the invention, a composition comprising gabapentin may be administered intracerebroventricularly to a patient. Administration of gabapentin directly to the cerebroventricles of a patient may allow for greater concentration of gabapentin in the local environment of the brain than intrathecal administration. Generally, intracerebroventricular administration is more difficult to perform than intrathecal. However, intrathecal administration of gabapentin may not always provide sufficient gabapentin concentration in the brain to treat epilepsy. Relative density of CSF to composition comprising gabapentin, location within the spinal canal of intrathecal delivery, degree of hydrophobicity, and other factors, such as described in, for example, copending application Ser. No. 10/745,731, entitled, METHOD FOR DELIVERING DRUG TO BRAIN VIAL THE SPINAL CANAL, filed on Dec. 23, 2003, which application is herby incorporated herein by reference., may play a role in determining whether sufficient concentrations of intrathecally administered gabapentin may reach a patient's brain to treat epilepsy. In addition to the spinal cord, it should be appreciated that the subarachnoid space of the brain may also be used for intrathecal gabapentin infusion in order to achieve higher levels of drug near an epileptic focus within the cerebral cortex. When intrathecal administration of gabapentin may produce insufficient concentrations of gabapentin in the brain, it may be desirable to administer gabapentin intracerebroventricularly. Regardless of the rational for administering gabapentin intracerebroventricularly, the invention contemplates such administration for the treatment of epilepsy.

Brain Tissue

According to an embodiment of the invention, a composition comprising gabapentin may be administered intraparenchymally to a patient's brain. As used herein, “intraparenchymal” administration means administration directly to brain tissue. Intraparenchymal administration may be directed to any brain region where gabapentin therapy may be effective to treat epilepsy. In an embodiment, gabapentin is administered intraparenchymally to a region of a patient's brain proximate to or within an area of an epileptic focus.

Delivery System

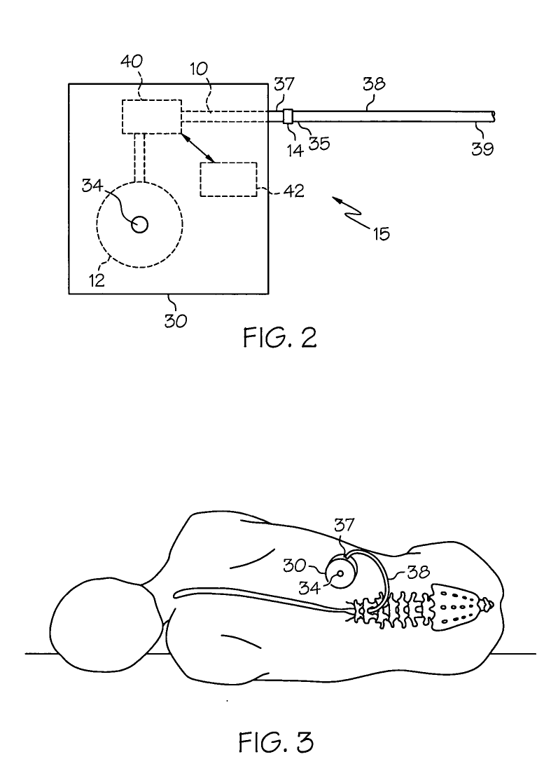

An embodiment of the invention provides a system for delivering to cerebrospinal fluid 6 or brain tissue of a patient a composition comprising gabapentin in an amount effective to treat epilepsy in the patient. Referring to FIG. 2, a system 15 for delivering a composition comprising gabapentin is shown. The system comprises a therapy delivery device 30. The device 30 comprises a pump 40 coupled to a reservoir 12 for housing a composition comprising a therapeutic agent, such as gabapentin. The system 15 further comprises a catheter 38. The catheter 38 comprises a proximal end 35 coupled to the pump 40 and a distal end 39 adapted for delivering the composition to a patient's cerebrospinal fluid 6 or brain tissue. It will be recognized that the catheter 38 may have one or more drug delivery regions along the length of the catheter 38 and that a drug delivery region may or may not be at the distal end 39 of the catheter 38. The therapy delivery device 30 may be implantable or may be an external device. The therapy delivery device 30 may have a port 34 into which a hypodermic needle can be inserted to inject a quantity of therapeutic agent into reservoir 12. The device 30 may have a catheter port 37, to which the proximal end 35 of catheter 38 may be coupled. The catheter port 37 may be coupled to pump 40 through an internal catheter 10. A connector 14 may be used to couple the catheter 38 to the catheter port 37 of the device 30. Device 30 may take the form of the device shown in U.S. Pat. No. 4,692,147 (Duggan), assigned to Medtronic, Inc., Minneapolis, Minn., commercially available as the Synchromed® infusion pump, which is incorporated by reference.

The therapeutic device 30, such as Medtronic's SYNCHROMED pump system, may be operated to discharge a predetermined dosage of the pumped fluid into the CSF 6 or brain of a patient. The therapeutic device 30 may contain a microprocessor 42 or similar device that can be programmed to control the amount of fluid delivery. The programming may be accomplished with an external programmer/control unit via telemetry. A controlled amount of fluid comprising therapeutics may be delivered over a specified time period. With the use of a therapeutic delivery device 30, different dosage regimens may be programmed for a particular patient. Additionally, different therapeutic dosages can be programmed for different combinations of fluid comprising therapeutics. Those skilled in the art will recognize that a programmed therapeutic device 30 allows for starting conservatively with lower doses and adjusting to a more aggressive dosing scheme, if warranted, based on safety and efficacy factors.

If it is desirable to administer more than one therapeutic agent, the composition within the reservoir 12 may contain a second, third, fourth, etc. therapeutic agent. Alternatively, the therapy delivery device 30 may have more than one reservoir 12 for housing additional compositions comprising a therapeutic agent. When the device 30 has more than one reservoir 12, the pump 40 may draw fluid from the one or more reservoirs 12 and deliver the drawn fluid to the catheter 38. The device 30 may contain a valve coupled to the pump 40 for selecting from which reservoir(s) 12 to draw fluid. Further, one or more catheters 38 may be coupled to the device 30. Each catheter 38 may be adapted for delivering a therapeutic agent from one or more reservoirs 12 of the device 30. A catheter 38 may have more than one lumen. Each lumen may be adapted to deliver a therapeutic agent from one or more reservoirs 12 of the pump 40. It will also be understood that more than one implantable device 30 may be used if it is desirable to deliver more than one therapeutic agent. Such therapy delivery devices, catheters, and systems include those described in, for example, copending application Ser. No. 10/245,963, entitled IMPLANTABLE DRUG DELIVERY SYSTEMS AND METHODS, filed on Dec. 23, 2003, which application is hereby incorporated herein by reference.

Referring to FIGS. 3, 4, and 5, a system or device 30 may be implanted below the skin of a patient. Preferably, the device 30 is implanted in a location where the implantation interferes as little as practicable with patient activity. Device 30 may be implanted subcutaneously in any medically acceptable area of the human body such as in a subcutaneous pocket located in the chest below the clavicle, in an abdomenal subcutaneous pocket, and the like.

According to an embodiment of the invention, distal end 39 of catheter 38 is positioned to infuse a fluid into a target area of CSF 6 of the patient. As shown in FIG. 3, catheter 38 may be positioned so that the distal tip 39 of catheter 38 is located in the subarachnoid space 3 of the spinal cord between the fifth lumbar and fifth thoracic vertebrae. It will be understood that the distal tip 39 can be placed in a multitude of locations to deliver a therapeutic agent into the cerebrospinal fluid 6 of the patient. Within the spinal cord, the distal tip 39 of the catheter 38 may be inserted, for example, in the subarachnoid space 3 between the fifth thoracic (T5) and the first cervical vertebrae (C1), in the subarachnoid space 3 between the fifth lumbar (L5) and fifth thoracic vertebrae (T5), in the subarachnoid space of the brain, etc. The location of the distal tip 39 of the catheter 38 may be adjusted to improve therapeutic efficacy. Administering a composition comprising gabapentin at a level in the spinal canal nearer the brain may result in increased concentrations of gabapentin in the brain. Alternatively, a composition comprising gabapentin may be administered directly into the cerebral ventricles. While device 30 is shown in FIG. 3, delivery of a composition comprising gabapentin into the CSF to treat epilepsy can be accomplished by injecting the therapeutic agent via port 34 to catheter 38.

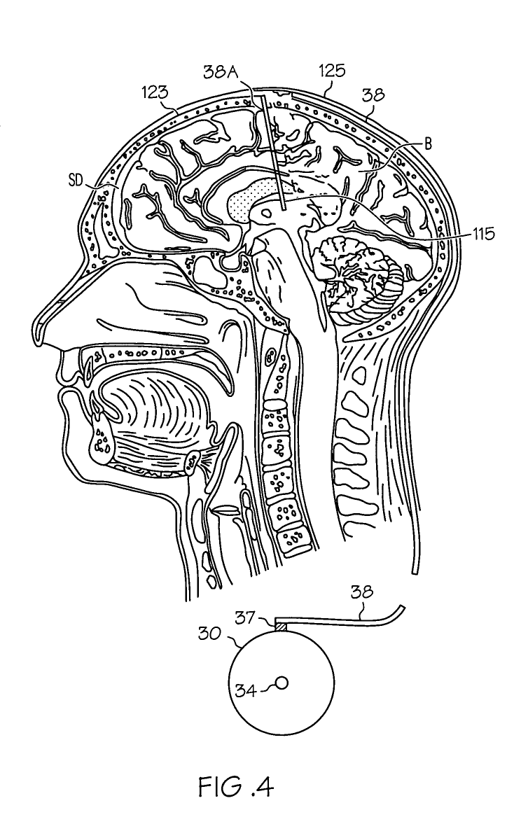

Referring to FIG. 4, a system for intraparenchymal or intracerebroventricular administration of a composition comprising gabapentin is shown. Device 30 and delivery system 15 may take the form of a device and system described in U.S. Pat. No. 6,042,579, entitled “Techniques for treating neurodegenerative disorders by infusion of nerve growth factors into the brain”, which patent is incorporated herein by reference in its entirety. As shown in FIG. 4, the distal end of catheter 38 may terminate in a cylindrical hollow tube 38A having a distal end 115 implanted into a portion of the brain by conventional stereotactic surgical techniques. The distal portion 115 may be implanted in the brain in any medically acceptable region. In an embodiment of the invention, the distal portion 115 is implanted in a region within or proximate to an epileptic focus. In an embodiment, portion 115 comprises details as described in U.S. application Ser. No. 08/430,960, now abandoned, entitled “Intraparenchymal Infusion Catheter System,” filed Apr. 28, 1995 in the name of Dennis Elsberry et al. and assigned to the same assignee as the present application, which application is herein incorporated by reference. Tube 38A may be surgically implanted through a hole in the skull 123 and catheter 38 may be implanted subcutaneously between the skull and the scalp 125 as shown in FIG. 4. Catheter 38 may be joined to implanted device 30 in the manner shown and may be secured to device 30 by, for example, securing catheter 38 to catheter port 37. In an embodiment, distal end 115 of cylindrical hollow tube 38A may be implanted in a ventricle of the brain. Alternatively, the distal tip may be located in the subdural area (SD) beneath the dura under the skull 123 but outside the brain B, and within the subarachnoid space. Catheter 38 may be divided into twin tubes 38A and 38B (not shown) that are implanted into the brain bilaterally. Alternatively, tube 38B (not shown) implanted on the other side of the brain may be supplied with drugs from a separate catheter 38 and device.

The delivery system 15 may include one or more sensor. The one or more sensors may detect an event associated with a seizure and may relay information regarding the detected event to processor 42 of device 30. Based on the sensed information, processor 42 may adjust parameters associated with therapy delivery to prevent or treat the seizure. The one or more sensors may be a sensor as described in, e.g., U.S. Pat. No. 5,978,702, entitled TECHNIQUES OF TREATING EPILEPSY BY BRAIN STIMULATION AND DRUG INFUSION, which patent is hereby incorporated herein by reference.

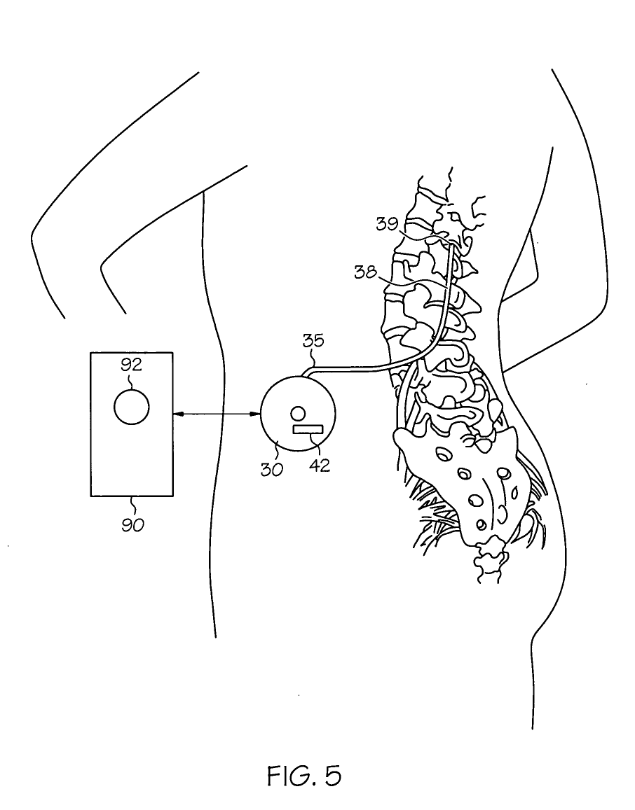

As shown in FIG. 5, a system for delivering therapeutic agent may include a patient-controlled activator 90, PCA. A PCA 90 may communicate with an implantable pump 40 to adjust the amount of therapeutic agent delivered. Communication between PCA 90 and implantable device 30 may be through any suitable means. In an embodiment, communication is through telemetry. Communication may be unidirectional; i.e., from PCA 90 to device 30, or bi-directional. PCA 90 may be a hand-held device. PCA may contain a button 92 or other suitable means for a patient to indicate a desire to alter amount of therapeutic agent delivered. Typically, a patient will depress button 92 or activate other suitable means to direct device 30 to deliver additional therapeutic agent, such as a composition comprising gabapentin. Generally, a pulse or short-term increase in infusion rate of therapeutic agent will occur as a result of the patient depressing the button 90. In an embodiment, a patient may place PCA 90 over skin in an area where device 30 is implanted. The amount and frequency of patient-controlled therapy administration may be limited by a physician or other health care provider by specifically programming the PCA 90 for a particular patient. Preferably, such programming controls would be inaccessible to the patient. It will be appreciated that a similar PCA 90 feature can be included in an external pump without the requirement of an additional device component. It will be further appreciated that while FIG. 5 depicts intrathecal administration, a PCA 90 may be used with intracerebroventricular, intraparenchymal, and other routes of administration in accordance with various embodiments of the invention.

Treatment of Epilepsy

An embodiment of the invention provides a method for treating epilepsy. In an embodiment, the method comprises delivering to cerebrospinal fluid (CSF) 6 of a patient a composition comprising gabapentin in an amount effective to treat epilepsy in the patient. A composition comprising gabapentin may be administered to a patient's CSF 6 in any medically acceptable manner, such as intrathecally, intracerebroventricularly, and the like. In an embodiment, the method comprises delivering to brain tissue of a patient a composition comprising gabapentin in an amount effective to treat epilepsy in the patient. Preferably the composition comprising gabapentin is delivered to a seizure-generating brain tissue. The composition may be administered intraparenchymally within or proximate to an epileptic focus. Direct administration of a composition comprising gabapentin to cerebrospinal fluid 6 or brain tissue of a patient in need thereof may result in elevated CNS concentrations of gabapentin relative to oral administration, and thus may result in increased efficacy in the treatment of epilepsy. In addition, direct administration to cerebrospinal fluid 6 may result in fewer or decreased side effects, even with increased CNS concentrations. It will be understood that the amount of gabapentin delivered or the location in which gabapentin is delivered may be adjusted based upon the presentation and severity of side effects in a patient. Side effects may be recognizable by the patient, a physician attending to the care of the patient, other care givers, and the like. A physician or other health care professional may adjust therapy parameters based on side effects. Side effects which may be associated with gabapentin include: somnolence, dizziness, ataxia, fatigue, motor weakness, nausea and/or vomiting.

Epilepsy, a seizure disorder, is “a recurrent, paroxysmal disorder of cerebral function characterized by sudden, brief attacks of altered consciousness, motor activity, sensory phenomena, or inappropriate behavior caused by excessive discharge of cerebral neurons” (The Merck Manual of Diagnosis and Therapy, 17th edition, section 14, chapter 172). According to an embodiment, the method of the invention may be useful for treating epilepsy regardless of the type of associated seizure. For example, the method may be useful for treating epilepsy associated with auras, simple-partial seizures, jacksonian seizures, complex partial seizures, generalized seizures, infantile spasms, absence seizures, generalized-tonic-clonic seizures, atonic seizures, myoclonic seizures, febrile-seizures, status epilepticus, epilepsia-partialis-continua, and the like, and combinations thereof. For additional information regarding epilepsy and associated seizures, see The Merck Manual of Diagnosis and Therapy, 17th edition.

In an embodiment, the invention provides a method for treating intractable epilepsy, which is defined herein as epilepsy that is either non-responsive or poorly responsive to treatment with currently approved, orally administered anti-epileptic agents. As many as 30% of epileptic patients do not respond or respond poorly to currently available orally administered anti-epileptic agents. Elevated CNS concentrations of gabapentin, due to administration directly to cerebral spinal fluid 6 or brain tissue, may prove more efficacious in the treatment of intractable epilepsy.

In an embodiment, a system for delivering gabapentin to cerebrospinal fluid 6 or brain tissue of a patient for the purposes of treating epilepsy includes a patient-controlled activator component 90 which allows the patient to increase the dose of gabapentin being administered by an implanted therapy delivery device 30. Because the patient may experience prodromal epileptic signs and symptoms, the patient may be the most appropriate person to assess an impending seizure and treat accordingly. The PCA component 90 may be activated by a patient and may interact with an implanted device 30. In an embodiment the PCA component 90 interacts with the implanted device 30 through telemetry. When the patient senses an imminent seizure, the patient may activate the PCA component 90 in an area near or over the skin where the device 30 is implanted. An additional amount (pulse or short-term increase in the infusion rate) of a composition comprising gabapentin may then be administered. In an embodiment, a similar PCA 90 feature is included in an external pump system without the requirement of an additional device component.

In an embodiment, the invention provides a method for treating epilepsy, where the method comprises administering a composition comprising gabapentin to a patient's CSF 6 or brain tissue and administering one or more additional epilepsy treating therapeutic agents to the patient. The one or more additional therapeutic agents may be administered in any medically acceptable manner. In an embodiment, the one or more additional therapeutic agents are administered directly to a patient's central nervous system, including directly to brain tissue or cerebrospinal fluid. The one or more additional therapeutic agents may be administered in the same manner as the composition comprising gabapentin. In an embodiment, the composition comprising gabapentin further comprises the one or more therapeutic agents. The one or more additional therapeutic agents are preferably administered at doses effective to treat epilepsy.

The one of more additional therapeutic agents may be any suitable agent effective for treating epilepsy. Non-limiting examples of classes of suitable antiepileptic agents are hydantoins, barbiturates, deoxybarbitures, iminostilbenes, succinimides, valproic acid, oxazolidinediones, benzodiazepines, and phenyltrizines. Non-limiting examples of suitable specific antiepileptic agents include phenyloin, mephenyloin, ethotoin, phenobarbitol, mephobarbitol, primodone, carbamazepine, ethosuximide, methsuximide, phensuximide, valproate, triemethadione, paramethadione, diazepam, clonazepam, midazolam, baclofen, thyrotropin-releasing hormone, adenosine and lamotrigine. In an embodiment, the one or more additional therapeutic agent includes one or more of baclofen, midazolam, and valproate Na.

Compositions

In an embodiment, the invention provides a method comprising administering to cerebrospinal fluid 6 of a patient a composition comprising gabapentin. As used herein, gabapentin refers to 1-(aminomethyl)cyclohexane acetic acid and pharmaceutically acceptable salts, solvates, hydrates, and polymorphs thereof. 1-(aminomethyl)cyclohexane acetic acid is a γ-aminobutyric acid (GABA) analogue with a molecular formula of C₉H₁₇NO₂ and a molecular weight of 171.24. 1-(aminomethyl)cyclohexane acetic acid is freely soluble in water and in both basic and acidic aqueous solutions. 1-(aminomethyl)cyclohexane acetic acid has the following structure:

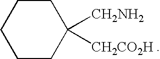

Gabapentin may be obtained from a variety of commercial sources, such as Shanghai Zhongxi International Trading Co., Shanghai, China; Hikal Limited, Bangalore, Karnaraka, India; Erregierre S.p.A., San Paolo d'Argon (BG), Italy; MediChem, SA, Sant Joan Despi (Barcelona), Spain; Ranbaxy Laboratories, New Delhi, India; Procos S.p.A., Cameri, Italy; Zambon Group, Milan, Italy; Hangzhuo Chiral Medicine Chemicals Co., Hangzhuo, China; InterChem Corporation USA, Paramus, N.J.; SST Corporation, Clifton, N.J.; Teva Pharmaceuticals USA, North Whales, Pa.; Plantex USA, Hakensack, N.J.; and Sigma-Aldrich, St. Louis, Mo., or an appropriate distributor. Alternatively, gabapentin may be synthesized and/or prepared as known in the art.

Any gabapentin composition suitable for administration to cerebrospinal fluid 6 or brain tissue may be used in a method according to the invention. Typically, the composition will be injectable. Injectable compositions include solutions, suspensions, and the like. Injectable solutions or suspensions may be formulated according to techniques well-known in the art (see, for example, Remington's Pharmaceutical Sciences, Chapter 43, 14th Ed., Mack Publishing Co., Easton, Pa.), using suitable dispersing or wetting and suspending agents, such as sterile oils, including synthetic mono- or diglycerides, and fatty acids, including oleic acid.

Solutions or suspensions comprising gabapentin may be prepared in water, saline, isotonic saline, phosphate-buffered saline, citrate-buffered saline, and the like and may optionally mixed with a nontoxic surfactant. Dispersions may also be prepared in glycerol, liquid polyethylene, glycols, DNA, vegetable oils, triacetin, and the like and mixtures thereof. Under ordinary conditions of storage and use, these preparations may contain a preservative to prevent the growth of microorganisms. Pharmaceutical dosage forms suitable for injection or infusion include sterile, aqueous solutions or dispersions or sterile powders comprising an active ingredient which powders are adapted for the extemporaneous preparation of sterile injectable or infusible solutions or dispersions. Preferably, the ultimate dosage form is a sterile fluid and stable under the conditions of manufacture and storage. A liquid carrier or vehicle of the solution, suspension or dispersion may be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. Proper fluidity of solutions, suspensions or dispersions may be maintained, for example, by the formation of liposomes, by the maintenance of the required particle size, in the case of dispersion, or by the use of nontoxic surfactants. The prevention of the action of microorganisms can be accomplished by various antibacterial and antifungal agents, for example, parabens, chlorobutanol, phenol, sorbic acid, thimerosal, and the like. In many cases, it will be desirable to include isotonic agents, for example, sugars, buffers, or sodium chloride. Prolonged absorption of the injectable compositions can be brought about by the inclusion in the composition of agents delaying absorption—for example, aluminum monosterate hydrogels and gelatin. Excipients that increase solubility, such as cyclodextrin, may be added.

Sterile injectable solutions may be prepared by incorporating a therapeutic agent in the desired amount in the appropriate solvent with various other ingredients as enumerated above and, as required, followed by sterilization. Any means for sterilization may be used. For example, the solution may be autoclaved or filter sterilized. In the case of sterile powders for the preparation of sterile injectable solutions, the preferred methods of preparation are vacuum drying and freeze-drying techniques, which yield a powder of the active ingredient plus any additional desired ingredient present in a previously sterile-filtered solution.

In an embodiment, a composition comprising gabapentin is an injectable solution comprising an aqueous solvent. The solvent may be water or saline. The saline may be 0.9% (w/v) saline or a solution where just enough sodium chloride is added to make the final solution isotonic. In an embodiment, the final solution has a pH between about 4 and about 9, between about 5 and about 7, between about 5.5 and about 6.5, or about 6. The pH may be adjusted with HCl or NaOH. Preferably, the final solution contains less than about 5% gabapentin lactam. In an embodiment, the final solution is essentially free of preservatives, buffers, or a combination thereof.

A composition comprising gabapentin according to an embodiment of the invention includes an amount of gabapentin effective to treat epilepsy when administered to a patient's cerebrospinal fluid 6. A composition comprising gabapentin according to an embodiment of the invention includes an amount of gabapentin effective to treat epilepsy when administered directly to a patient's brain tissue. When the composition is a solution or suspension, the gabapentin may be present in the composition at any concentration sufficient to treat epilepsy. In an embodiment, gabapentin is present in a solution or suspension at a concentration between about 0.1 mg/mL and about 100 mg/mL. In an embodiment, gabapentin is present in a solution or suspension at a concentration between about 10 mg/mL and about 90 mg/mL. In an embodiment, gabapentin is present in a solution or suspension at a concentration between about 20 mg/mL and about 80 mg/mL. In an embodiment, gabapentin is present in a solution or suspension at a concentration of about 80 mg/mL. In an embodiment, a composition comprises between about 10 mg/ml and about 50 mg/ml gababentin. For example, the composition may comprise between about 20 mg/ml and 40 mg/ml, or about 30 mg/ml.

In an embodiment, an injectable composition comprising gabapentin is administered to cerebrospinal fluid 6 of a patient in a daily dose of between about 0.1 mg and about 200 mg. In an embodiment, gabapentin is administered in a daily dose of between about 1 mg and about 150 mg. In an embodiment, gabapentin is administered in a daily dose of between about 2 mg and about 60 mg. In an embodiment, gabapentin is administered in a daily dose of greater than about 25 mg. In an embodiment, gabapentin is administered in a daily dose of less than about 25 mg. For example, gabapentin may be administered at a daily dose of between about 0.1 mg and about 10 mg, between about 0.1 mg and 5 mg, between about 0.1 mg and 2 mg, between about 0.1 and 1 mg, between about 0.1 and 0.5 mg, or about 0.2 mg. It will be understood that daily dose requirements may be adjusted to account for variability in CSF volume, CSF production rates, and rate of clearance of gabapentin from the CSF. One of skill in the art will understand that such variability may be due in part to, e.g., gender and/or age. In an embodiment, the composition comprising gabapentin is administered intrathecally. An implantable therapy delivery device 30 may be used for intrathecal administration. When a therapy delivery device 30 is used, a composition comprising gabapentin may be infused into a patient's cerebrospinal fluid 6 through continuous infusion or as pulses over time. The rate of the infusion and the frequency and duration of the pulses may be controlled by a microprocessor 42 in the device 30.

A composition comprising gabapentin may be co-administered with one or more additional therapeutic agents for the treatment of epilepsy. The one or more additional therapeutic agents may be administered in a separate composition from the composition comprising gabapentin, or the composition comprising gabapentin may further comprise one or more additional therapeutic agents. Preferably, the one or more additional theraputic agent is an antiepileptic agent. Non-limiting examples of classes of suitable antiepileptic agents are hydantoins, barbiturates, deoxybarbitures, iminostilbenes, succinimides, valproic acid, oxazolidinediones, benzodiazepines, and phenyltrizines. Non-limiting examples of suitable specific antiepileptic agents include phenyloin, mephenyloin, ethotoin, phenobarbitol, mephobarbitol, primodone, carbamazepine, ethosuximide, methsuximide, phensuximide, valproate, triemethadione, paramethadione, diazepam, clonazepam, midazolam, baclofen, thyrotropin-releasing hormone, adenosine and lamotrigine.

In an embodiment, the one or more additional therapeutic agent includes one or more of baclofen, midazolam, and valproate Na. Baclofen may be present in a fluid composition at any suitable concentration, such as between about 50 μg/ml to about 3000 μg/ml, between about 100 μg/ml to about 2500 μg/ml, or between about 1000-2000 μg/ml. Baclofen may be administered directly to a patient's cerebrospinal fluid 6 or brain tissue at any daily dose effective for treating epilepsy. Examples of suitable daily doses include between about 50 μg/day and about 1500 μg/day, between about 100 μg/day and about 1250 μg/day, and between about 500 μg/day and about 1000 μg/day. Midazolam may be present in a fluid composition at any suitable concentration, such as between about 1 mg/ml to about 5 mg/ml. Midazolam may be administered directly to a patient's cerebrospinal fluid 6 or brain tissue at any daily dose effective for treating epilepsy, such as between about 0.1 mg/day and about 5 mg/day. Valproate Na may be present in a fluid composition at any suitable concentration, such as between about 1 mg/ml to about 100 mg/ml. Valproate Na may be administered directly to a patient's cerebrospinal fluid 6 or brain tissue at any daily dose effective for treating epilepsy, such as between about 5 mg/day and about 100 mg/day.

It will be understood that the use of combination therapy may provide for increased efficacy while allowing for use of lower doses of each agent in the combination therapy (relative to if any agent were used alone in monotherapy). Decreased doses of each individual agent in combination therapy may limit side effects associated with any one of the individual agents.

The following patent applications are generally relevant to injectable gabapentin and its use:

U.S. patent application Ser. No. ______, entitled INTRATHECAL GABAPENTIN FOR TREATMENT OF PAIN, filed on even date herewith, and having Attorney Docket No. P-20216.00;

U.S. patent application Ser. No. ______, entitled INJECTABLE GABAPENTIN COMPOSITIONS, filed on even date herewith, and having Attorney Docket No. P-20904.00;

U.S. patent application Ser. No. ______, entitled PROCESS FOR PRODUCING INJECTABLE GABAPENTIN COMPOSITIONS, filed on even date herewith, and having Attorney Docket No. P-20907.00; and

U.S. patent application Ser. No. ______, entitled PUMP SYSTEMS INCLUDING INJECTABLE GABAPENTIN COMPOSITIONS, filed on even date herewith, and having Attorney Docket No. P-20906.00.

All patents, patent applications, technical papers, and other publications cited herein are hereby incorporated by reference herein, each in its respective entirety. As those of ordinary skill in the art will readily appreciate upon reading the description herein, at least some of the compositions, devices and methods disclosed in the patents and publications cited herein may be modified advantageously in accordance with the teachings of the present invention.

1. A system comprising:
a pump coupled to a reservoir;
a catheter coupled to the pump and adapted for delivering a therapeutic agent to a cerebrospinal fluid of a patient; and
an injectable composition comprising gabapentin housed in the reservoir and deliverable through the catheter in an amount effective to treat epilepsy in the patient when administered to the cerebrospinal fluid of the patient.

2. The system of claim 1, wherein the composition comprises between about 0.1 mg/mL and about 100 mg/mL gabapentin.

3. The system of claim 2, wherein the composition comprises between about 10 mg/mL to about 80 mg/mL gabapentin.

4. The system of claim 2, wherein the solution comprises about 80 mg/mL gabapentin.

5. The system of claim 2, wherein the solution comprises between about 20 mg/ml and about 40 mg/ml gabapentin.

6. The ststem of claim 2, wherein the solution comprises between about 30 mg/ml gabapentin.

7. The system of claim 1, wherein the composition is an aqueous solution.

8. The system of claim 7, wherein the aqueous solution comprises sodium chloride.

9. The system of claim 8, wherein the solution comprises about 0.9% (w/v) sodium chloride.

10. The system of claim 8, wherein the solution comprises an amount of sodium chloride such that the solution is substantially isotonic with the cerebrospinal fluid.

11. The system of claim 7, wherein the solution has a pH between about 4 and about 9.

12. The system of claim 11, wherein the solution has a pH between about 5, and about 7.

13. The system of claim 12, wherein the solution has a pH between about 5.5 and about 6.5.

14. The system of claim 7, wherein the solution comprises essentially no preservatives.

15. The system of claim 14, wherein the solution comprises essentially no buffers.

16. The system of claim 1, wherein the composition comprises less than about 5% (w/v) gabapentin lactam.

17. The system of claim 7, wherein the solution has a pH between about 5.5 and about 6.5, and comprises essentially no preservatives, essentially no buffers, and less than about 5% (w/v) gabapentin lactam.

18. The system of claim 1, wherein the pump is an implantable pump.

19. The system of claim 1, wherein the composition further comprises one or more additional anti-epileptic agent.

20. The system of claim 19, wherein the one or more additional antiepileptic agent is selected from the group consisting of:
a hydantoin, a barbiturate, a deoxybarbiture, an iminostilbene, a succinimide, valproic acid, an oxazolidinedione, a benzodiazepine, and a phenyltrizins.

21. The system of claim 19, wherein the one or more additional antiepileptic agent is selected from the group consisting of:
phenyloin, mephenyloin, ethotoin, phenobarbitol, mephobarbitol, primodone, carbamazepine, ethosuximide, methsuximide, phensuximide, valproate, triemethadione, paramethadione, diazepam, clonazepam, midazolam, baclofen, thyrotropin-releasing hormone, adenosine, and lamotrigine, or a pharmacologically acceptable salt thereof.

22. The system of claim 1, wherein the composition further comprises baclofen.

23. The system of claim 22, wherein the composition comprises between about 50 μg/ml and about 3000 μg/ml baclofen.

24. The system of claim 1, wherein the composition further comprises midazolam.

25. The system of claim 24, wherein the composition comprises between about 1 mg/ml and about 5 mg/ml midazolam.

26. The system of claim 1, wherein the composition further comprises valproate Na.

27. The system of claim 26, wherein the composition comprises between about 1 mg/ml and about 100 mg/ml valproate Na.

28. A system comprising:
a pump coupled to a reservoir;
a catheter coupled to the pump and adapted for delivering a therapeutic agent to a brain tissue of a patient; and
an injectable composition comprising gabapentin housed in the reservoir and deliverable through the catheter in an amount effective to treat epilepsy in the patient when administered to the brain tissue of the patient.

29. A method for treating a epilepsy in a patient in need thereof, the method comprising:
administering to a cerebrospinal fluid of the patient a composition comprising gabapentin in an amount effective to treat epilepsy in the patient, wherein the composition is administered by a pump system.

30. The method of claim 29, wherein the composition is administered to the patient's cerebrospinal fluid.

31. The method of claim 30, wherein the composition is administered to the patient's spinal cord.

32. The method of claim 30, wherein the composition is administered by infusing gabapentin into the subarachnoid space around the brain.

33. The method of claim 30, wherein the composition is administered intracerebroventricularly.

34. The method of claim 29, wherein the composition is administered to directly to the patient's brain tissue.

35. The method of claim 29, further comprising selecting a patient with a history of seizures selected from the group consisting of:
auras, simple-partial seizures, jacksonian seizures, complex partial seizures, generalized seizures, infantile spasms, absence-seizures, generalized-tonic-clonic-seizures, atonic seizures, myoclonic seizures, febrile-seizures, status-epilepticus, epilepsia-partialis-continua, and combinations thereof.

36. The method of claim 29, wherein the epilepsy is intractable epilepsy.

37. The method of claim 29, wherein gabapentin is administered at a daily dose of between about 0.1 mg and about 200 mg.

38. The method of claim 29, wherein gabapentin is administered at a daily dose of between about 1 mg and about 150 mg

39. The method of claim 38, wherein gabapentin is administered at a daily dose of between about 2 mg and about 60 mg.

40. The method of claim 29, wherein gabapentin is administered at a daily dose of greater than about 25 mg.

41. The method of claim 29, wherein gabapentin is administered at a daily dose of less than about 25 mg.

42. The method of claim 41, wherein gabapentin is administered at a daily dose of between about 0.1 mg and about 10 mg.

43. The method of claim 29, wherein the pump is an implantable pump.

44. The method of claim 43, wherein the patient controls the amount of gabapentin administered.

45. The method of claim 44, wherein the patient controls the amount of gabapentin administered by way of a patient-controlled activator.

46. The method of claim 29, further comprising administering to the patient one or more additional anti-epileptic agent.

47. The method of claim 46, wherein the one or more additional anti-epileptic agent is selected from the group consisting of:
a hydantoin, a barbiturate, a deoxybarbiture, an iminostilbene, a succinimide, valproic acid, an oxazolidinedione, a benzodiazepine, and a phenyltrizins.

48. The method of claim 46, wherein the one or more additional antiepileptic agent is selected from the group consisting of:
phenyloin, mephenyloin, ethotoin, phenobarbitol, mephobarbitol, primodone, carbamazepine, ethosuximide, methsuximide, phensuximide, valproate, triemethadione, paramethadione, diazepam, clonazepam, midazolam, baclofen, thyrotropin-releasing hormone, adenosine and lamotrigine, or a pharmacologically acceptable salt thereof.

49. The method of claim 46, wherein the one or more additional anti-epileptic agent is administered to the patient's cerebrospinal fluid or brain tissue.

50. The method of claim 49, wherein at least one of the one or more additional antiepileptic agent is baclofen or a pharmacologically acceptable salt thereof.

51. The method of claim 50, wherein the baclofen or the pharmacologically acceptable salt thereof is administered at a daily dose of between about 50 μg and about 1500 μg.

52. The method of claim 49, wherein at least one of the one or more additional antiepileptic agent is midazolam or a pharmacologically acceptable salt thereof.

53. The method of claim 52, wherein the midazolam or the pharmacologically acceptable salt thereof is administered at a daily dose of between about 0.1 mg and about 5 mg.

54. The method of claim 49, wherein at least one of the one or more additional antiepileptic agent is valproate Na.

55. The method of claim 55, wherein the valproate Na is administered at a daily dose of between about 5 mg and about 100 mg.

56. A method for treating epilepsy in a patient in need thereof, the method comprising:
administering to a cerebrospinal fluid of the patient a composition comprising gabapentin in an amount effective to treat epilepsy in the patient, wherein gabapentin is administered at a daily dose of greater than about 25 mg and wherein the patient experiences substantially no somnolence, dizziness, ataxia, or motor weakness due to the gabapentin.

57. A method for treating epilepsy in a patient in need thereof, the method comprising:
administering to a brain tissue of the patient a composition comprising gabapentin in an amount effective to treat epilepsy in the patient, wherein gabapentin is administered at a daily dose of greater than about 25 mg and wherein the patient experiences substantially no somnolence, dizziness, ataxia, or motor weakness due to the gabapentin.

58. A method for preparing a system of claim 1, comprising adding the injectable composition to the reservoir.

59. A method for preparing a system of claim 28, comprising adding the injectable composition to the reservoir.
